(19) United States
(12) Patent Application Publication
(10) Pub. No.: US 2002/0082588 A1
(43) Pub. Date: Jun. 27, 2002

(54) Laparoscopic applicator

(75) Inventors: Michael McMahon, Leeds (GB); Edward Almond, Leeds (GB); Peter Moran, Leeds (GB); Jean-Marie Gilbert, Sur Seine (FR); Stuart Moran, Leeds (GB)

Correspondence Address: BEYER WEAVER & THOMAS LLP, P.O. BOX 778, BERKELEY, CA, 94704-0778, US

(73) Assignee: Genzyme SA

(21) Appl. No.: 09/863,807

(22) Filed: May 22, 2001

(30) Foreign Application Priority Data
Jun. 1, 2000 (GB) 0013279.5

(51) Int. Cl. A61B 17/00

(52) U.S. Cl. 606/1; 604/19

(57) ABSTRACT

A laparoscopic apparatus for inserting and applying a sheet of surgical material comprising:

a handle;

a sleeve extending forwardly of the handle;

a divided spindle comprising a plurality of elongate members forming jaws which are moveable between an open position in which a sheet of surgical material may be placed between or removed from the jaws and a closed position wherein te sheet may be engaged between the jaws and furled on the spindle, the members being biassed towards the open position;

the elongate members extending through said sleeve, and be slidably within sleeve md the elongate members be urged into te closed position as the spindle is withdrawn into the sleeve;

the sleeve and the elongate members extending hugh a protective outer tube so that the elongate members and a sheet furled there may be reversibly withdrawn into a forward end of the outer tube.

This invention relates to apparatus for inserting and applying sheets of surgical material for example absorbable adhesion barrier sheet, in laparoscopic surgery. This invention also relates to a method of use of such apparatus and to the use of the apparatus in application of sheets of surgical material.

Adhesion barriers are moisture sensitive and must be protected from contact with moist surfaces during application A surgical instrument for application of adhesion barrier sheets is disclosed in WO92/06638 which discloses a rod around which the sheet may be furled for insertion through a trocar sleeve into the body cavity. EP-A-535506 discloses an instrument wherein a rod-like carrier has a slit to receive the barrier sheet, and a cylindrical sleeve into which the rod and furled sheet may be stowed during insertion into the body cavity. WO97/36622 discloses apparatus for endoscopic insertion of a sheet wherein the sheet is engaged by pivotal jaws which may fit inside: a bore during insertion into the body cavity.

According to the present invention laparoscopic apparatus for inserting and applying a sheet of surgical material comprises:

a handle;

a sleeve extending forwardly of the handle;

a divided spindle comprising a plurality of elongate members forming jaws which arc moveable between an open position in which a sheet of surgical material may be placed between or removed from the jaws and a closed position wherein the sheet may be engaged between the jaws and furled on the spindle, the members being biassed towards the open position;

the elongate members Ceding through said sleeve, and being slidable within the sleeve, and the elongate members being urged into the closed position as the spindle is withdrawn into the sleeve;

the sleeve and the elongate members extending through a protective outer tube so that the elongate members and a sheet furled thereon may be reversibly withdrawn into a forward end of the outer tube.

The laparoscopic applicator of this invention has the advantage of a simple construction which is reliable and easy to use. Use of pivotally connected jaws operated by a push rod is avoided. Furthermore the apparatus may include a simple manual actuation arrangement which is easy and convenient to use.

In a preferred embodiment of the invention a rearward end of the sleeve has a first knob adapted to engage a user's hand;

the rearward end of the spindle having a second knob;

so means being arranged to bias the first and second knobs away from each other to urge the jaws into the closed position;

the first knob being manually moveable towards te second knob against the action of the sag to open the jaws.

This preferred actuation arrangement allows a surgeon to place the first knob in the palm of his hand and engage the second knob between two fingers or between finger and thumb. The apparats may be rotated freely using the wrist or by rolling between the finger and thumb, avoiding the limitations imposed by a pistol grip-like arrangement,

The first and second knobs are preferably generally axially symmetrical. The first knob may be provided with a freely rotatable end cap to facilitate rotation of the apparatus in contact with a surgeons palm.

In particularly preffered embodiments a latch is provided to lock the sleeve within the outer tube. The latch maybe adapted to lock te sleeve in any position within the outer tube. Alternatively latch may be adapted to lock the sleeve in predetermined locations. In one embodiment the latch comprising a locking plate having an aperture with a dimension which reduces from a first dimension greater than the diameter of the sleeve to a second dimension which is an interference fit on the sleeve or adapted to engage a recess in te sleeve, the sleeve passing through the aperture and the locking plate bed moveable between an unlocked position wherein the sleeve can move freely and a locked position wherein the sleeve is prevented from movement

A lever may be provided to facilitate operation of the locking plate. In a preferred embodiment a spring serves to bias the lever into the locked position.

In preferred embodiments the apparatus includes an end cap on the forward end of one or more of the elongate member the end cap being adapted to engage the forward end of the outer tube to prevent ingress of moisture. The end cap may be provided with an 0 ring or seal The external surface of the forward end of the outer tube may have a plurality of circumferential grooves or rings to impede flow of moisture to the interior of the tube or onto the spindle during use. The end cap may comprise a rounded or domed structure having a diameter selected to cooperate closely with the open end of the outer tube, The spindle preferably comprises two elongate members extending forwardly from a shaft portions a it elongate member extending axially of the shaft and a second elongate member joining the first member at the forward end of the shafts He forward end of the second member extending away from the first member. This may provide a generally Y-shaped configuration.

It is particularly preferred that the first elongate member may carry an end cap. This arrangement is particularly advantageous because the end cap may be provided with a socket into which the free, forward end of the second member may be received when the spindle is in the closed position. This prevents the fee end of the second member from become fouled during insertion of the sheet into the body cavity.

Opening and closing of the elongate members of the spindle may be facilitated by provision of one or sore radially outwardly extending cam surfaces thereon. Advantageously one or both of the elongate members may have outwardly bowed portions located so that withdrawal of the spindle into the sleeve causes the bowed or otherwise outwardly extending portions to engage he cad of the sleeve, causing the members to close during a short axial movement of the spindle.

The present invention also provides use of the apparatus as disclosed above in application of a surgical sheet, preferably a bioadhesion sheet. The invention also provides a method of application of such a surgical sheet using the apparatus as disclosed above.

The invention is further described by means of example but not in any limitative sense with reference to the accompanying drawings of which:

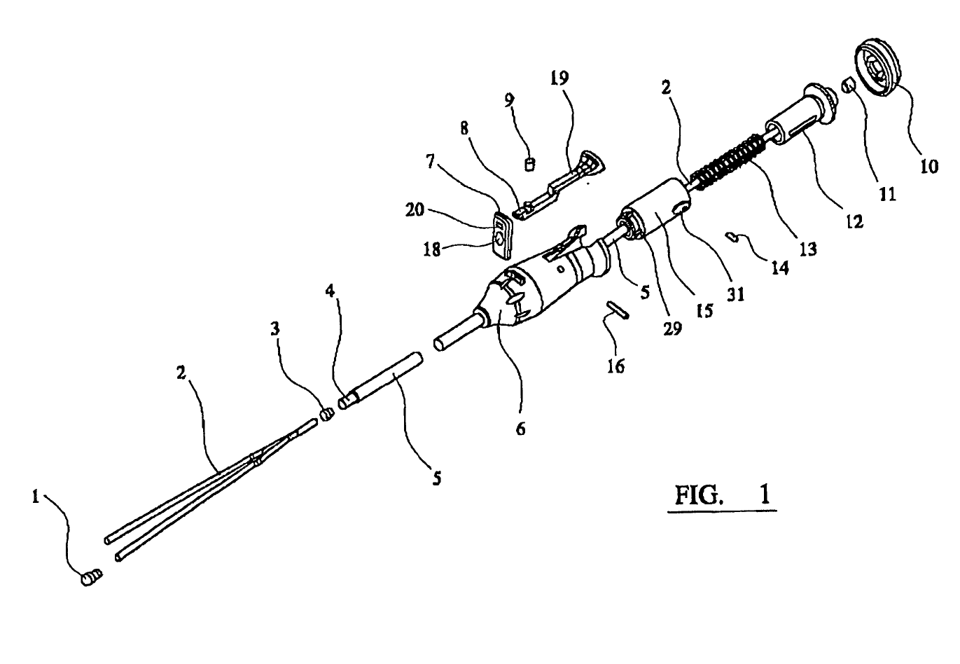

FIG. 1 is a partially exploded view of a laparoscopic applicator in accordance with this invention;

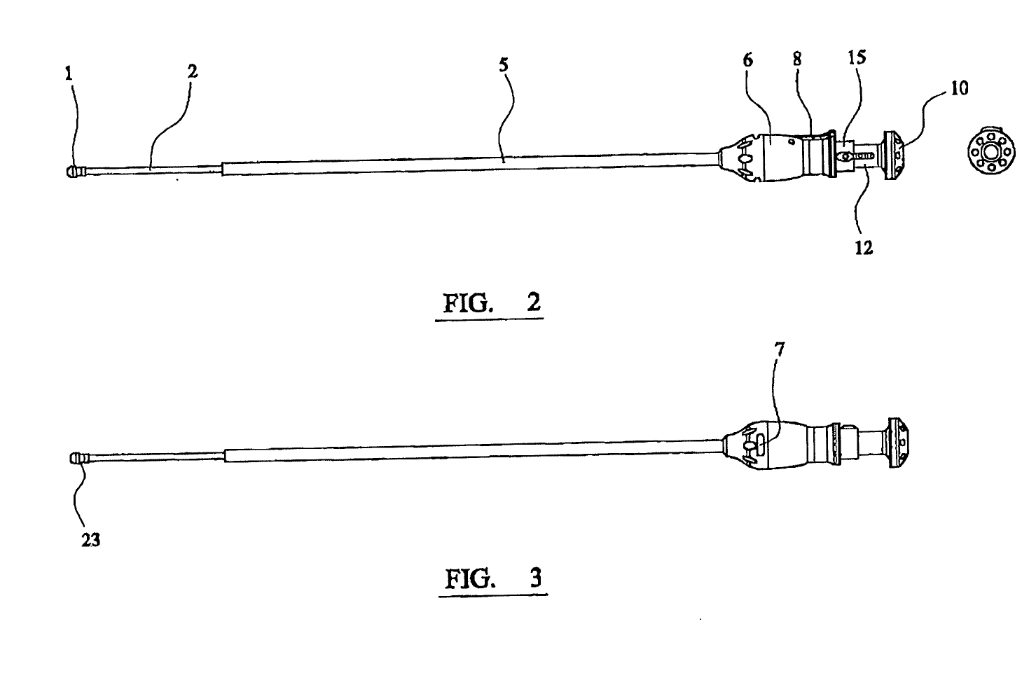

FIG. 2 is a plan view of the applicator;

FIG. 3 is a side elevation of the applicator;

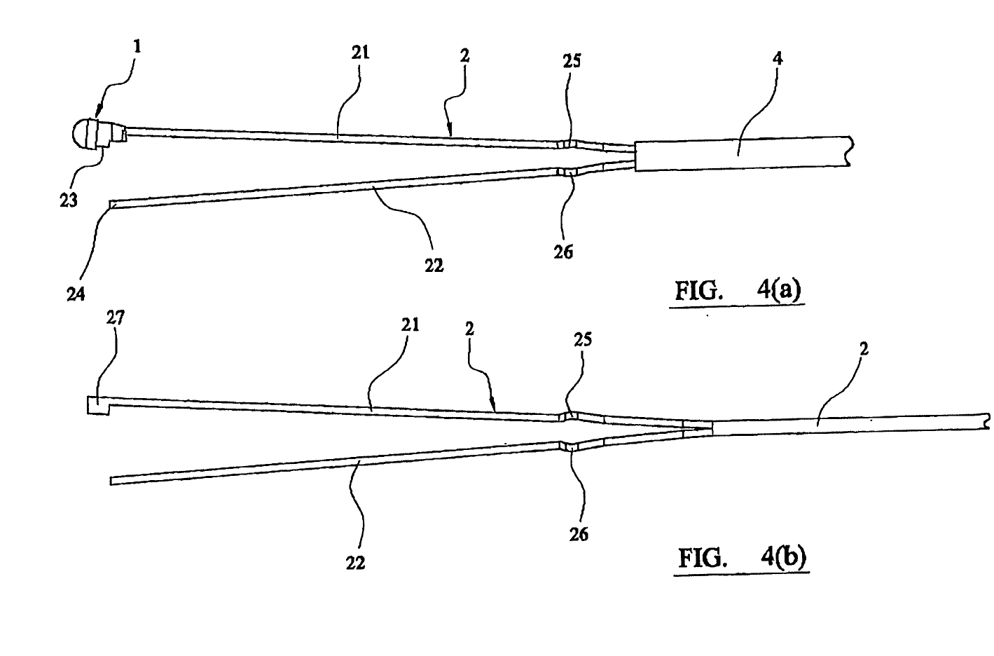

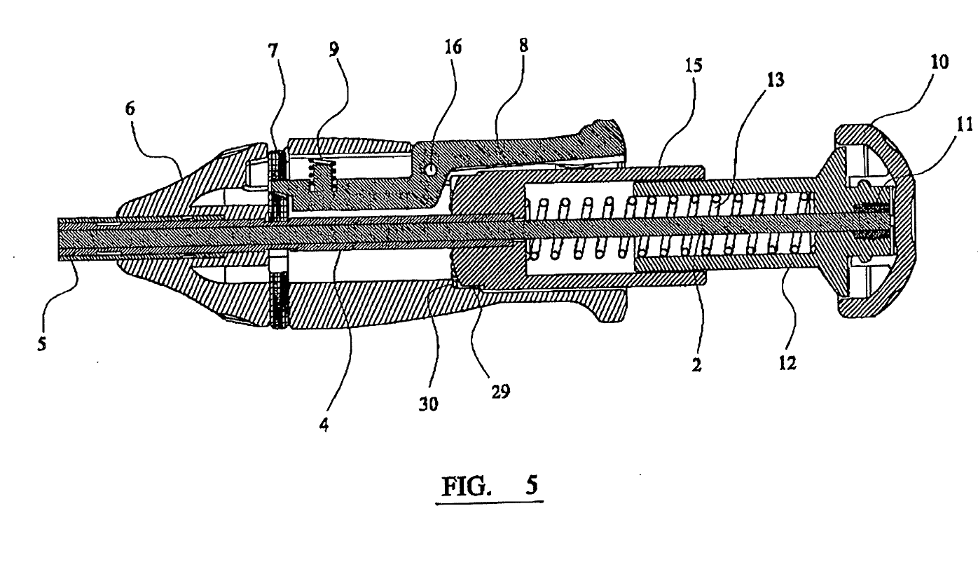

FIG. 4 illustrates the elongate members;

FIG. 5 is a cross-sectional view of the rear end of the applicator,

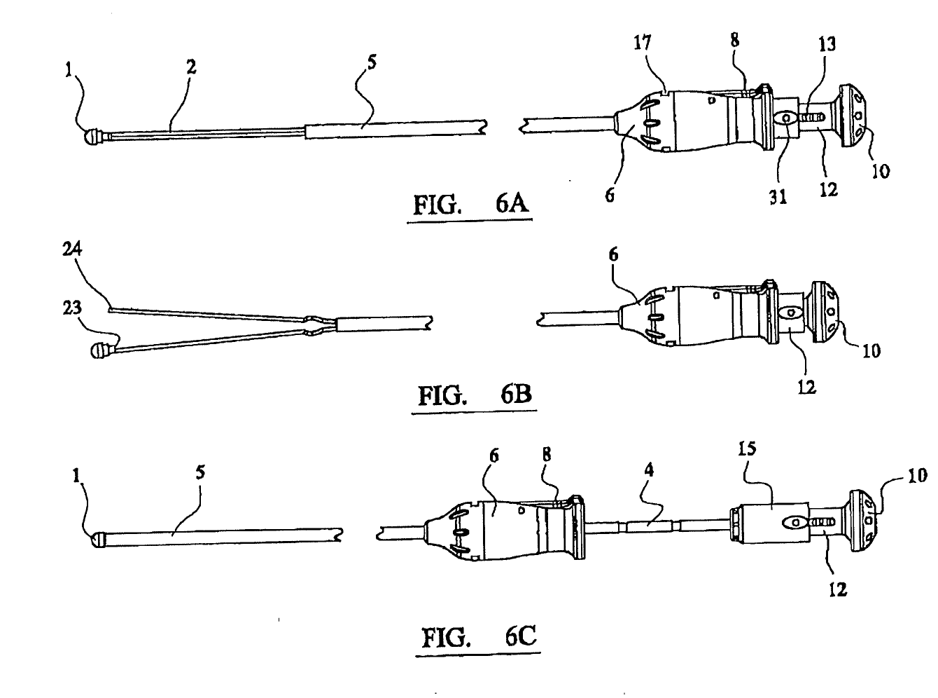

FIGS. 6a to 6c show the applicator in different stages of use; and

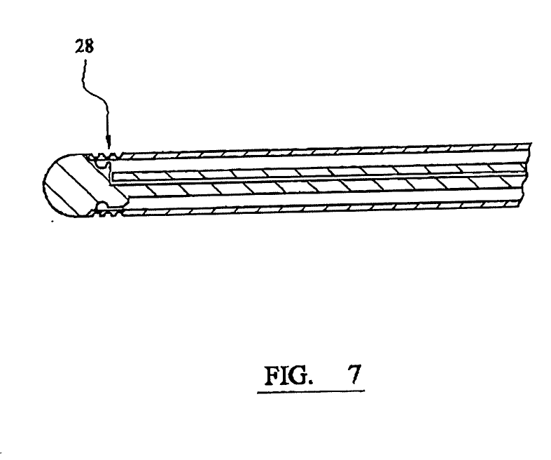

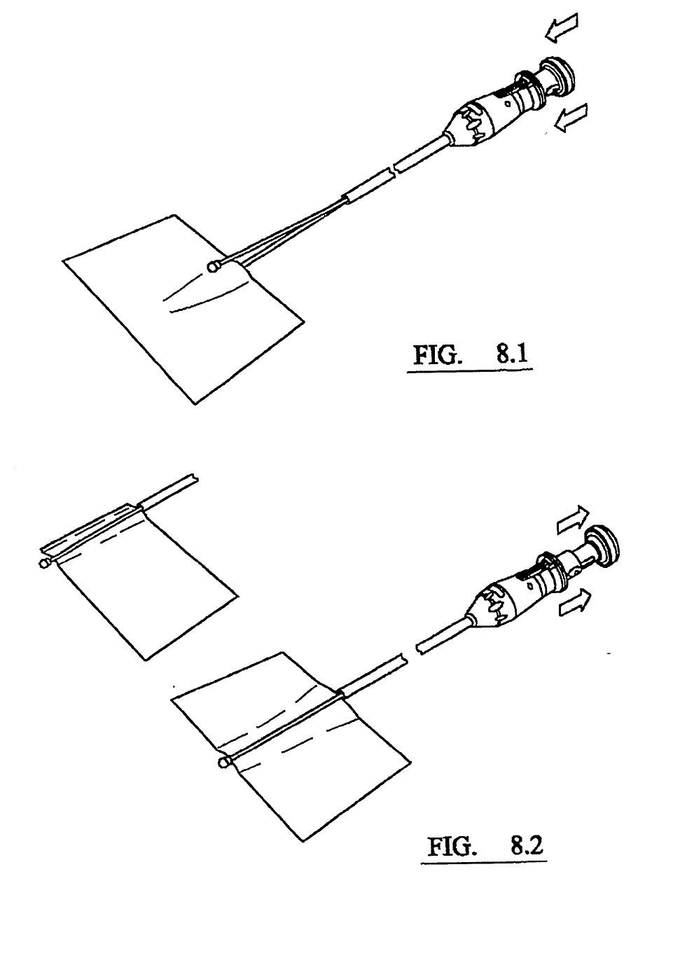

FIG. 7 is a detail of one end of the outer tube;

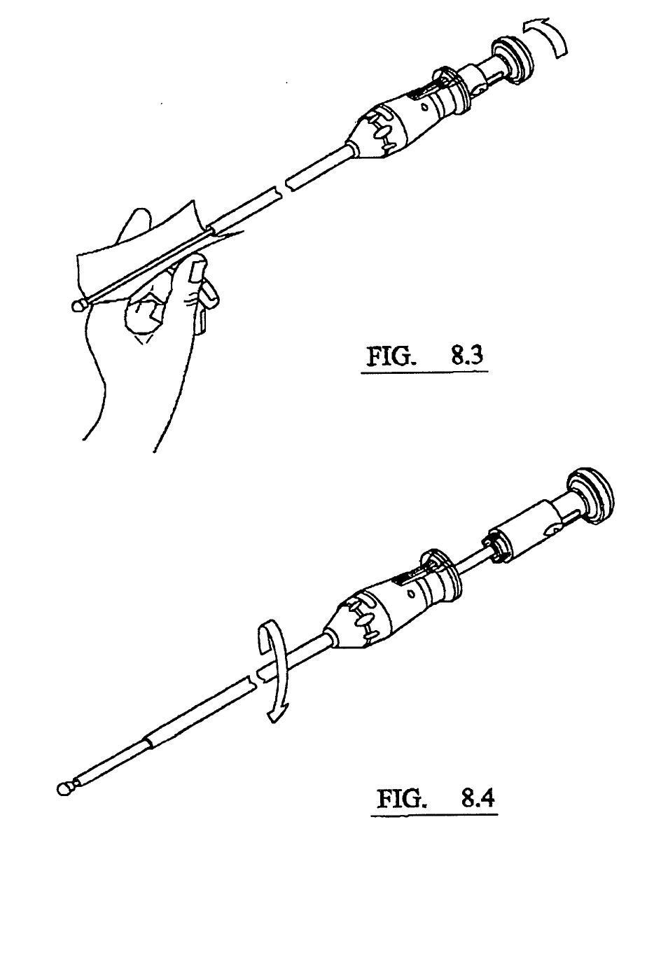

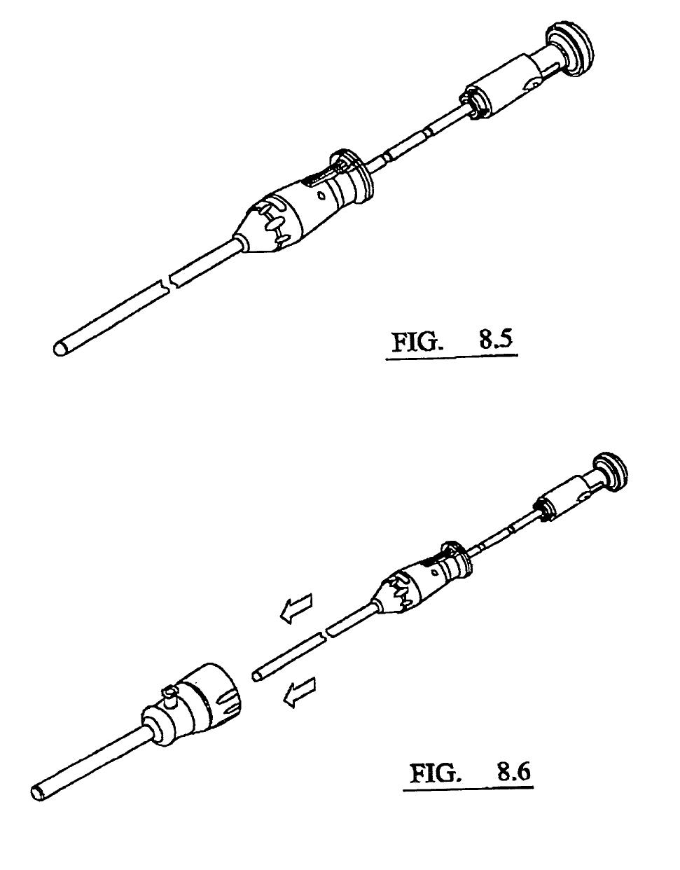

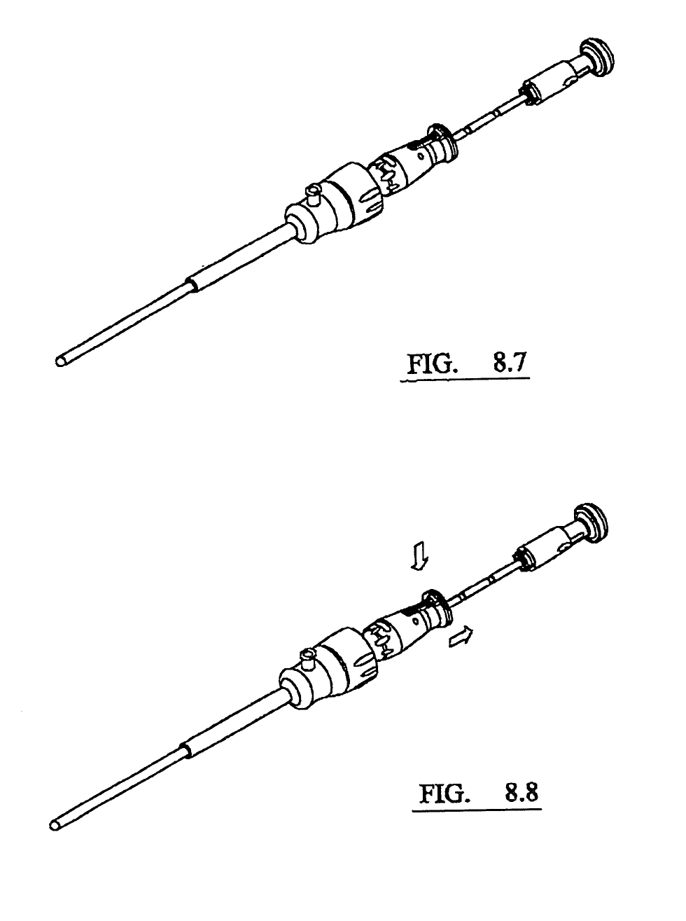

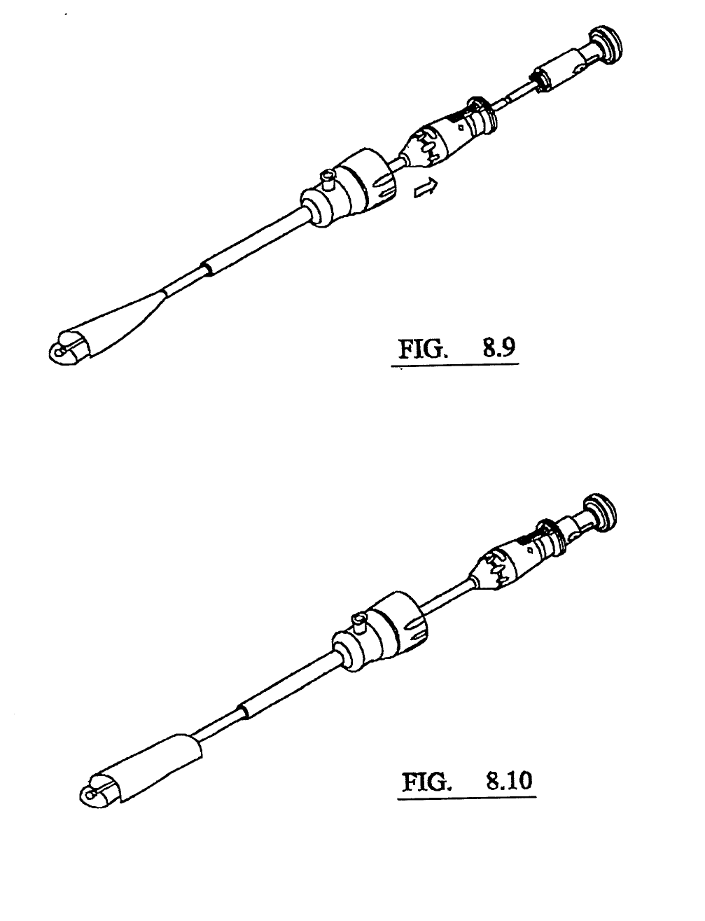

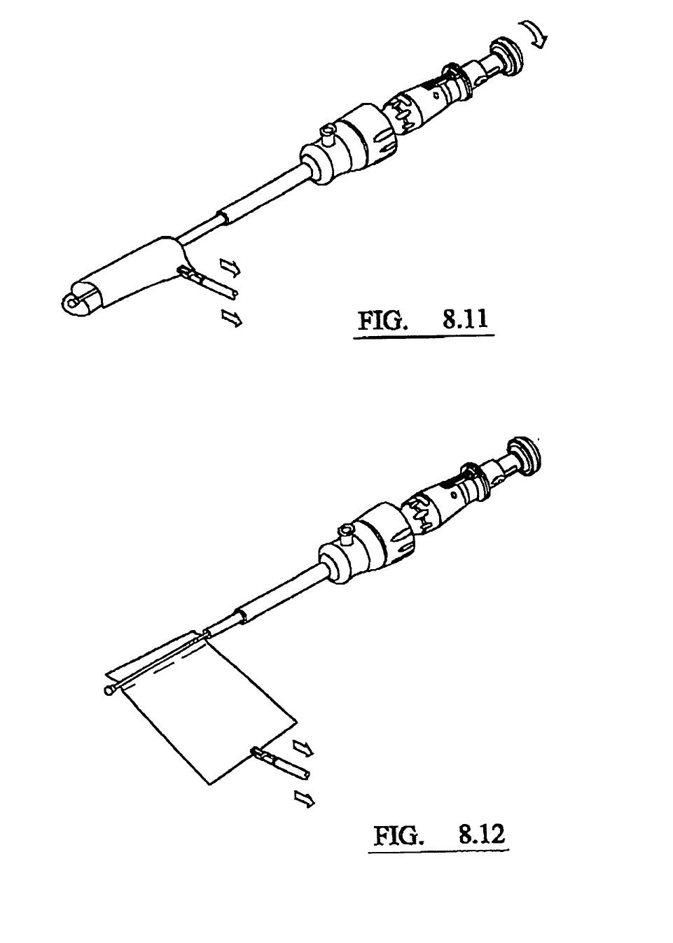

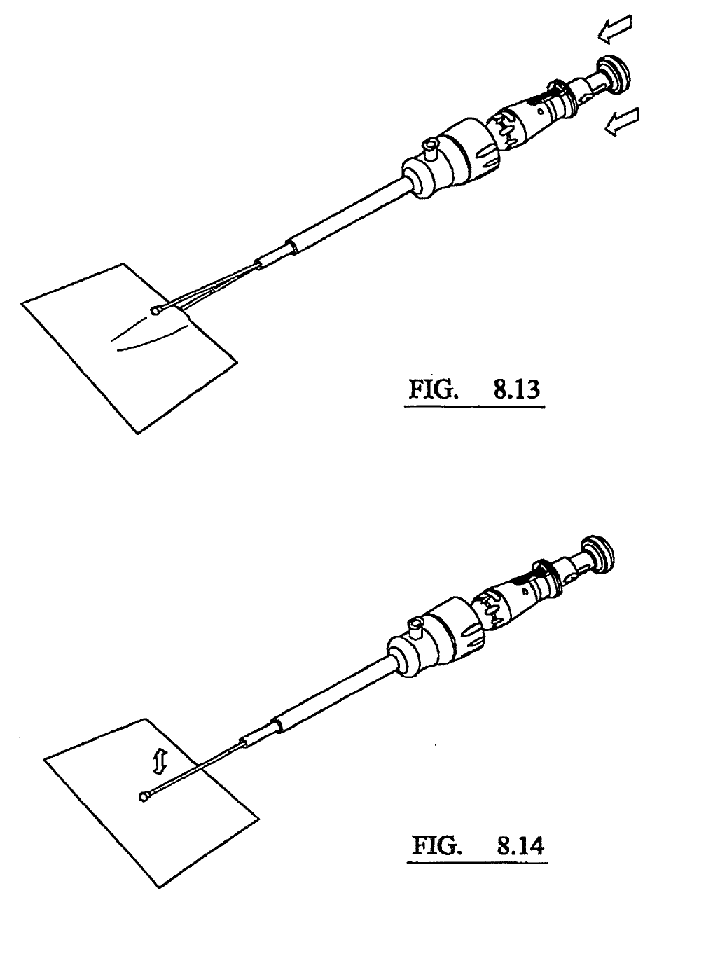

FIG. 8.1 to 8.14 illustrate use of the applicator in application of a surgical sheet.

The same reference numerals are used to denote the same components in differ The laparoscopic applicator illustrated in FIGS. 1 to 6 comprises a sleeve 4 having a bush 3 into which a divided spindle 2 comprising two (or more) elongate members is inserted. An end cap 1 is secured to the forward end of an elongate member 2. The sleeve 4 is freely slidable within an outer tube 5. A main body 6 formed of moulded plastics material is secured to the outer tube 5. The rearward end of the sleeve 4 has a closing tube knob 15 and the rearward end of the divided spindle 2 is secured in a spindle knob 12 by use of a tracking device 11, a helical spring 13 serving to urge the knobs I and 12 apart so that the divided spindle 2 is withdrawn into the sleeve 4. A rely rotatable palm knob 10 is carried on an integral bet and allows the apparatus to be freely rotated when the knob 10 rests in the palm of a surgeon's hand. A locking plate 7 slidably within an aperture 17 of the main body 6 includes an aperture 18 through which the sleeve 4 In a first embodiment he aperture 18 has a reducing dimension so that in a first position of the locking plate 7 the sleeve 4 can freely move within the outer tube 5, and in a second position the dimension of the aperture 18 is smaller so that the sleeve 4 is engaged in an interference fit to prevent movement thereof. In an alternative embodiment he sleeve 4 may have a pliability of recesses adapted to receive and engage upper and lower uses of the aperture 18 (as shown), hereby looking movement of the shaft. This gives an advantage that a locking position will maintain the membrane in a filled position yet allows the membrane to be seen and contacted by auxiliary instrumentation.

A lever 8 mounted on a pin 16 engages a socket 20 of the locking plate 7. A spring 9 serves to urge the locking plate downwardly (as shown) so that the sleeve 4 is locked unless the actuator 19 on the lever is depressed against the action of the sag 9. The assembled applicator is shown in FIGS. 2 and 3.

FIG. 4a shows the sleeve 4 carrying the divided spindle 2. FIG. 4b also shows a detail of the divided spindle 2 in which two elongate members 21, 22 are joined together. The forward end of the first elongate member 21 has a lug 27 to which an end cap 1 is fitted. The end cap 1 has a forwardly facing domed or rounded portion to facilitate safe insertion into the body cavity, A socket 23 is dimensioned to receive the forward end 24 of the second elongate member 22. This prevents the elongate members 21, 22 from becoming separated during use.

The elongate members 21, 22 have outwardly bowed portions 25, 26. These abut and engage the opening of the sleeve 4 as the divided spindle 2 is retracted into the sleeve 4. Closure of the elongate members 21, 22 occurs quick during a short axial movement of the sleeve 4. In an alternative embodiment projections or other cam surfaces may be formed in place of the bowed portions 25, 26.

FIG. 7 shoes the distal end of te outer tube 5 provided with a plurality of circumferential grooves 28 defining ribs to impede flow of moisture from the end of the tube during use.

Referring now to FIGS. 8.1 to 8.14, these show successive stages in the carrying out of a surgical method utilizing the preferred embodiment of the invention.

FIG. 8.1 depression of the palm knob 10 and the spindle knob assembly towards knob 15 to open up the divided spindle 2. This picks up te membrane using divided spindle 2.

FIG. 8.2 shows the position of the membrane equidistantly about the divided spindle 2, or biassed towards one side of membrane, and release of palm bob 10 and spindle bob 12 assembly thereby closing divided spindle 2.

FIG. 8.3 shows grasping the membrane between finger and thumb and rotation of the handle assembly to furl the membrane around the divided spindle 2.

FIG. 8.4 shows depression of lever 8, thereby disengaging the locking plate 7, from the recess sleeve 4, and slide and rotate outer tube 5 towards end cap 1 whilst maintaining the membrane in a furled condition.

In FIG. 8.5 locking plate 7 will automatically engage in a suitable recess in sleeve 4, thereby locking outer tube 5 over the membrane and the divided spindle 2. This protects the membrane from damage and moisture.

FIG. 8.6 shows insertion of the applicator into suitable cannula.

In FIG. 8.8, depress lever 8, thereby releasing locking plate 7 from mating recess in sleeve 4, and with drawing outer tube 5, by grasping main body 6 in a proximal direction.

The FIG. 8.9 drawback outer tube 5 until looking plate 7 engages in a recess in sleeve 4, such that the membrane remains fled, but can be seen with the tail end located and grasped as required.

In FIG. 8.10, depressed lever 8, thereby releasing locking plate 7 from its mating recess in sleeve 4, and withdraw outer tube 5 until locking plate 7 engages in rearmost position.

FIG. 8.11, when locking plate 7 engages in its rearmost position, external engagement positions 29 on outer tube knob 15 engage into mating internal features 30 within body 6 and allow the entire handle to rotate as one unit. Grasp film using instrument with jaws made from a non-stick metal, and rotate handle to rotate the membrane

In FIG. 8.12, use the applicator and additional instrument to position membrane within abdominal cavity.

In FIG. 8.13, depress palm b 10 ad spindle knob 12 assembly towards knob 15 to open the divided spindle 2, thereby releasing the membrane.

In FIG. 8.14, tamp the membrane in poison using non-stick end cap 1.

The following further advantageous features are set out below:

1. A plurality of external engagement features 29 on outer tube knob 15 engage at a finite number of positions into mating internal features 30 within main body 6 when locking plate 7 is engaged in appropriate recess wit sleeve 4. This ensures that te handle behaves as one for rotational purposes.

2. Alignment feature 31 indicates orientation of divided spindle 2.

3. End cap 1 is a non-stick material.

4. Sleeve 4 has a locking position that maintains film in furled condition yet allows membrane to be seen and contacted with grasping forceps.

What I claim is:

1. A laparoscopic apparats for inserting and applying a sheet of surgical material comprising:
a handle;
a sleeve extending forwardly of the handle;
a divided spindle comprising actuality of elongate members forming jaws which arc moveable between an open position in which a sheet of surgical material may be placed between or removed from the jaws and a closed position wherein 1he sheet may be engaged between the jaws and furled on the spindle, the members being biassed towards toe open position;
the elongate members extending through said sleeve, and being slidable within the sleeve and the elongate member being urged into the closed position as the spindle is withdrawn into the sleeve;
the sleeve and the elongate members extending through a protective outer tube so that the elongate members and a sheet furled thereon may be reversibly drawn into a forward end of the outer tube.

2. Apparatus as claimed in claim 1, wherein the rearward end of the sleeve has a first knob adapted to engage a user's hand;
the rearward end of the spindle having a second knob;
spring means being arranged to bias the first and second knobs away from each other to urge the jaws into the closed position;
the first knob being manually moveable towards the second knob against the action of the spring to open the jaws.

3. Apparatus as claimed in claim 2, wherein the firsthand second knobs are generally axially symmetrical.

4. Apparatus as claimed in claim 2, wherein the first knob is provided with an axially rotatable end cap.

5. Apparatus as claimed in claim 1, wherein a latch is provided to lock the sleeve within the outer tube.

6. Apparatus as claimed in claim 5, wherein the latch comprises a locking plate having an apparatus with a dimension which reduces from a fist dimension greater than the diameter of the sleeve to a second dimension which is an interference fit on the sleeve or adapted to engage a recess in the sleeve the sleeve passing through the aperture and the locking plate being moveable between an unlocked position wherein the sleeve can move freely and a locked position wherein the sleeve is prevented from movement

7. Apparatus as claimed in claim 6, wherein to facilitate operation of te locking plate.

8. Apparats as claimed in claim 7, wherein a spring serves to bias the lever into the locked position.

9. Apparatus as claimed in claim 1, including an end cap on the forward end of one or more of the elongate members, the end cap being adapted to engage the forward end the outer tube

10. Apparatus as claimed in claim 1, wherein the spindle comprises two elongate members extending forwardly from a shaft portion, a first elongate member extending axially of the shaft and second elongate member joining the first member at the forward end of the a the forward end of the second member extending away first the member.

11. Apparatus as claimed in claim 10, wherein the first elongate member carries an end cap.

12. Apparatus as claimed in claim 11, wherein the end cap is provided with a socket into which the free forward of the second member may be received when the spindle is in the closed position.

13. Apparatus as claimed in claim 10, wherein one or both of the elongate members may have outwardly bowed portions located so that withdrawal of te spindle into the sleeve cause the bowed or otherwise outwardly extending portions to engage the end of the sleeve, causing the members to close during a short axial movement of te spindle.

14. Use of apparatus as claimed claim 1in application of a surgical sheet.

15. A method of application of a surgical sheet using the apparatus as claimed in claim 1.
